(19) United States
(12) Patent Application Publication
(10) Pub. No.: US 2005/0037092 A1
(43) Pub. Date: Feb. 17, 2005

(54) Vascularization therapy by highly concentrated carbonated warm water bath

(75) Inventors: Hiroki Sakakibara, Chita-gun (JP); Ken Ooyachi, Owariasahi-shi (JP); Hiroaki Matsubara, Toyonaka-shi (JP)

Correspondence Address: Kendrew H. Colton;FITCH, EVEN, TABIN & FLANNERY, Suite 401L, 1801 K Street, N.W., Washington, DC, 20006-1201, US

(73) Assignees: Mitsubishi Rayon Co., Ltd., Tokyo (JP); Mitsubishi Rayon Engineering Co., Ltd., Tokyo (JP)

(21) Appl. No.: 10/676,270

(22) Filed: Oct. 2, 2003

(30) Foreign Application Priority Data
Apr. 1, 2003 (JP) P2003-098136

(51) Int. Cl. A61K 33/00

(52) U.S. Cl. 424/700

(57) ABSTRACT

A treatment method capable of regenerating new blood vessels at an affected site of a peripheral blood vessel that is highly safe with respect to the human body and is easily performed. More specifically, a vascularization therapy that increases the number of newly formed blood vessels of an affected site by immersing in carbonated warm water having a carbon dioxide concentration of 700 ppm or more and a water temperature of 33 to 42° C. A vascularization therapy that increases the number of vascular endothelial cells in tissue of an affected site by 1.5 times or more and that increases the number of endothelial precursor cells in the peripheral blood at an affected site by 1.1 times or more by immersing the affected site of a peripheral blood vessel in the aforementioned carbonated warm water.

### BACKGROUND OF THE INVENTION

1. Field of the Invention

The present invention relates to vascularization therapy using a highly concentrated carbonated warm water bath.

2. Description of Related Art

Since carbonated springs are thought to demonstrate superior effects on peripheral blood circulation failure due to their potent vasodilation action, they are widely used for therapy and hydropathic treatment. Although carbonated naturally flowing springs have been used in the past, due to the development of superior artificial carbonated spring production methods, artificial carbonated spring therapy has currently come to be widely used in the field of internal medicine. The vasodilation action of carbon dioxide is thought to occur by the mechanism described below.

Among the energy obtained by metabolism in the body, energy consumed directly in muscles and organs at that time is in the form of thermal energy generated during conversion of ATP (adenosine triphosphate) to ADP (adenosine diphosphate). Glycogen is oxidized by way of pyruvic acid and the TCA cycle, and the energy generated at that time is used as an energy source when returning the converted ADP to ATP. When glycogen is oxidized, simultaneous to consumption of oxygen, H₂O and carbon dioxide are released as metabolites, after which they are expelled outside the body by the blood in the form of exhaled gas from the lungs.

Within the body, various regulatory mechanisms are provided so as to ensure a stable supply of this required energy. A particularly important regulatory mechanism is the regulation of blood flow that serves as a transport path for substances required for metabolism. The amount of carbon dioxide present in blood and tissues has various effects on this regulatory mechanism of blood flow volume. Normally, an increase in the amount of carbon dioxide means a sthenia of metabolism (consumption of a large amount of energy). In order to compensate for this, blood vessels are dilated, or in other words, blood flow volume is increased, by several regulatory mechanisms to promote the supply of oxygen and discharge of carbon dioxide .

Carbon dioxide is easily absorbed percutaneously. Consequently, carbon dioxide, which is inherently the end product of metabolism, can be artificially increased in the tissues by absorbing carbon dioxide percutaneously from a carbonated water bath. As a result, blood vessels can be dilated and blood flow volume can be increased. As a result of this action, blood vessels are temporarily dilated. Alternatively, dysfunctional finer vessels can be opened again to increase blood flow volume or allow blood to enter peripheral tissues. This technique for alleviating symptoms is actively used in the form of carbonated springs therapy.

However, the vasodilation action brought about by carbonated water baths dissipates after several minutes. Thus, the aforementioned therapy utilizes the temporarily vasodilation action and increased blood flow volume brought about by carbon dioxide . The aforementioned therapy is limited its application to alleviation of pain in combination with temporary thermotherapy, supportive treatment of wound sites through increased blood flow, or reduction of risk and rehabilitation of heart disease utilizing lower blood pressure (reduction in vascular resistance) brought about by dilation of peripheral vessels.

In addition, there are also treatment methods other than the use of carbon dioxide that obtain similar effects by administration of a vasodilator such as prostaglandin E1 into the body. Although these treatment methods are able to sustain vasodilation action depending on the dosage, they are unable to heal hematogenous disorders themselves.

On the other hand, among serious peripheral circulatory diseases such as arteriosclerosis and diabetes, there are many cases in which blood vessels themselves irreversibly lose their inherent function, thereby essentially eliminating the significance of administration of vasodilators. There is therefore a need for a therapeutic method that is capable of regenerating blood vessels themselves instead of temporarily increasing blood flow volume.

The body is provided with a function that repairs blood vessels when they have been damaged or during defective circulation. This function is promoted by several tissue growth factors, and research is currently being conducted on therapy that promotes the regeneration of blood vessels by injecting a protein such as HGF (hepatocyte growth factor), VEGF (vascular endothelium growth factor) or FGF (fibroblast growth factor), or their genes, directly into the affected site. Therapy has also been proposed in which vascular endothelium precursor cells and bone marrow mononuclear cells that secrete the aforementioned growth factors are collected from the bone marrow, purified and then transplanted to the affected area disclosed in the papers as follows;
Tateishi-Yuyama, E., Matsubara, H., Murohara, T., Ikeda, U., Shintani, S., Masaki, H., Kishimoto, Y., Yoshimoto, K., Akashi, H., Shimada, K., Iwasaka, T. and Imaizumi, T.: Therapeutic angiogenesis for patients with extremity ischemia by autologous transplantation of bone marrow cells: a pilot study and a randomized controlled trial, Lancet, 360, 427-435 (2002).
Iba, O., Matsubara, H., Nozawa, Y, Fujiyama, S., Amano, K., Mori, Y., Kojima, H. and Iwasaka, T.: Angiogenesis by implantation of peripheral blood mononuclear cells and platelets into ischemic extremities, Circulation, 106, 2019-2025 (2002).

However, in order to administer the required growth factors and genes directly to the affected area in these treatment methods, it is necessary to capture and concentrate growth factor present only in minute amounts in the body, or synthesize genes that express them and insert the gene into a virus or other vector, to prepare in the form of a preparation. Moreover, administration to the affected area typically requires injection to the affected area at several tens of locations by injection of highly concentrated growth factor and so forth. In addition, since these growth factors are also presumed to act in other ways in addition to vascular regeneration, their adverse side effects are not insignificant in the body. In the case highly concentrated growth factor administered by injection has dispersed throughout the body by traveling in the blood, there is the possibility of it affecting the entire body.

In consideration of the problems of the prior art, an object of the present invention is to provide a therapeutic method which not only increases blood flow volume at an affected site of a peripheral blood vessel, but also is capable of regenerating the decreased or lost function of blood vessels, having a high degree of safety with respect to the body and being able to be performed easily.

### SUMMARY OF THE INVENTION

A first aspect of the present invention is a vascularization therapy that increases the number of newly formed blood vessels by performing a step in which an affected site of a peripheral blood vessel is immersed in carbonated warm water having a carbon dioxide concentration of 700 ppm or more and a water temperature of 33 to 42° C.

A second aspect of the present invention is a vascularization therapy that increases the number of vascular endothelial cells in tissue of an affected site by 1.5 times or more by performing a step in which an affected site of a peripheral blood vessel is immersed in carbonated warm water having a carbon dioxide concentration of 700 ppm or more and a water temperature of 33 to 42° C.

A third aspect of the present invention is a vascularization therapy that increases the number of endothelial precursor cells in the peripheral blood by 1.1 times or more by performing a step in which an affected site of a peripheral blood vessel is immersed in carbonated warm water having a carbon dioxide concentration of 700 ppm or more and a water temperature of 33 to 42° C.

The vascularization therapy of the present invention not only increases blood flow volume at an affected site of a peripheral blood vessel, but is also able to restore lost vascular function by the method of the present invention. Moreover, the method of the present invention has a high degree of safety with respect to the human body, and can be performed easily.

### BRIEF DESCRIPTION OF THE DRAWINGS

FIG. 1 is a graph showing the ameliorative effects on blood flow in a mouse ischemic lower extremity by a carbonated warm water bath.

FIG. 2 is a graph showing the increase in the number of new blood vessels in mouse ischemic lower extremity skeletal muscle by a carbonated warm water bath.

FIG. 3 is a graph showing the increase in the number of vascular endothelial cells in mouse ischemic lower extremity skeletal muscle by a carbonated warm water bath.

FIG. 4 is a graph showing the increase in the number of vascular endothelial precursor cells in mouse peripheral blood by a carbonated warm water bath.

### DETAILED DESCRIPTION OF THE INVENTION

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

The concentration of carbon dioxide in the carbonated warm water used in the method of the present invention is 700 ppm or more.

The amount of carbon dioxide percutaneous absorption from a carbonated warm water bath increases proportionately to the concentration of carbon dioxide dissolved in the carbonated warm water. In the case of simply applying to a wound site or being a supportive therapy of thermotherapy, carbon dioxide should be made to act in the vicinity of the skin surface. Thus, the effective minimum concentration of carbon dioxide is about 400 ppm in the case of healthy individuals.

However, in the vascularization therapy of the present invention, since it is necessary to allow the carbon dioxide to penetrate to deeper tissue. The effective minimum concentration is required to have a higher concentration, and that concentration is 700 ppm or more. If the concentration of carbon dioxide is less than 700 ppm, although vasodilation action is demonstrated, vascularization action is not obtained. The concentration of carbon dioxide is preferably from 1000 to 1400 ppm. If the concentration of carbon dioxide is within the aforementioned range, adequate vascularization effects are obtained, excessive irritation of the skin and tissue is not imparted, thereby making this preferable.

There are no particular restrictions on the method for achieving the carbon dioxide concentration as described above. Examples of such methods include a method in which carbon dioxide gas bubbles are generated and dissolved in warm water by a chemical reaction, a method in which carbon dioxide is dissolved by introducing warm water into a pressure tank filling with carbon dioxide gas (e.g., production of carbonated warm water using the Model AM2100 manufactured by Trautwein GmbH), and a method in which carbon dioxide is dissolved by introducing warm water and carbon dioxide gas into a gas-permeable hollow fiber membrane (e.g., production of carbonated warm water using the Model MRE-SPA-MD manufactured by Mitsubishi Rayon Engineering Co., Ltd.). Among these, it is preferable to use a carbonated warm water production device that uses the method in which carbon dioxide is dissolved by introducing warm water and carbon dioxide gas into a gas-permeable hollow fiber membrane since carbonated warm water having a carbon dioxide concentration of 1000 ppm or more can be produced easily and at an accurate concentration.

The saturated dissolved concentration of carbon dioxide in warm water at atmospheric pressure is 1150 ppm to 950 ppm at 33 to 42° C. Although varying according to individual differences and the presence of disease, the physiological action of carbon dioxide is enhanced concentration-dependently up to about 1400 ppm. As carbon dioxide dissolved to super-saturation easily decreases to the saturated concentration at atmospheric pressure. Carbon dioxide is more preferably replenished into the warm water continuously during the course of treatment for the purpose of maintaining the super-saturated concentration at all times.

The water temperature of the carbonated warm water used in the method of the present invention is from 33 to 42° C., and more preferably from 35 to 39° C. near body temperature. If the water temperature is lower than 33° C., vascularization action is unable to be adequately obtained, and it becomes difficult to immerse the affected area for the required amount of time since the carbonated water feels cold to the patient. If the water temperature is higher than 42° C., metabolism is accelerated excessively which has a detrimental effect on tissue regeneration, while also making it difficult to immerse the affected area for the required amount of time since the carbonated water feels hot to the patient.

In the present specification, the affected site of a peripheral blood vessel refers to a site at which there is an inadequate supply of blood to the tissue due to a peripheral vascular disease, and/or to a site at which peripheral vascular function has decreased or been lost due to ischemia.

Examples of peripheral circulatory disorders for which the vascularization therapy of the present invention can be used preferably include ischemic diseases caused by arteriosclerosis or thrombosis, diseases caused by peripheral circulatory disorders due to diabetes, and circulatory disorders of peripheral blood vessels caused by Buerger's disease, Raynaud's disease or collagen diseases.

There are no particular restrictions on the affected site of peripheral blood vessels. Typically, there are many cases having for the affected site an upper extremity or lower extremity. Among these, the affected site is below the knee or a pedal in the majority of cases, and since there is no effective treatment method, the vascularization therapy of the present invention can be used particularly preferably in these cases.

One aspect of the present invention is a vascularization therapy comprising increasing the number of new blood vessels at an affected site by performing a step in which the affected site of a peripheral blood vessel is immersed in the aforementioned carbonated warm water.

Among serious peripheral circulatory diseases caused by arteriosclerosis, diabetes and so forth, blood vessels themselves irreversibly lose their inherent function, and there are many cases in which the administration of vasodilators becomes essentially meaningless. Thus, there is a desire for a treatment method that makes it possible to regenerate lost blood vessels themselves rather than temporarily increasing blood flow volume. The method of the present invention is extremely effective since it is able to not only act to increase blood flow, but also increase the number of new blood vessels by easy method.

Increases in the number of new blood vessels at an affected site can be measured in the manner described below.

An increase in the number of new blood vessels brought about by the present invention is the result of the action of carbon dioxide absorbed percutaneously, and since this action is manifested particularly strongly near the skin, can be confirmed by measuring tissue blood flow volume with a laser-Doppler blood flowmetry microvascular that measures the blood flow volume on finer vessels near the skin. Although there is invasion into the body, increase of the number of new blood vessels is possible to confirm to measure by X-ray angiography involving the injection of a contrast medium. The number of blood vessels which there is blood flow can be measured by sampling tissue and identifying blood vessels in that tissue by staining the vessels using alkaline phosphatase (ALP), which is an enzyme secreted from blood vessels into the blood, as a marker. Increases in the number of viable blood vessels in tissue at an affected site can also be confirmed by determining the ratio of the number of vessels through which there is blood flow to the number of muscle fibers.

A second aspect of the present invention is a vascularization therapy that increases the number of vascular endothelial cells in the tissue at an affected site by 1.5 times or more by performing a step in which an affected site of a peripheral blood vessel is immersed in the aforementioned carbonated warm water. Since a large number of vascular endothelial cells indicate a high degree of vascularization, the increase in the number of vascular endothelial cells is more preferably 2.0 times or more.

Increases in the number of vascular endothelial cells that compose blood vessels can be confirmed by immunostaining vascular endothelial cells using von Willebrand factor (vWF), which specifically bonds to vascular endothelial cells, as a marker, and measuring the ratio of the number of vascular endothelial cells to the number of muscle fibers of tissue at the affected site.

A third aspect of the present invention is a vascularization therapy that increases the number of vascular endothelial precursor cells in the peripheral blood at an affected site by 1.1 times or more by immersing an affected site of a peripheral blood vessel in the aforementioned carbonated warm water. Since a larger number of vascular endothelial precursor cells indicates a more potent capacity to regenerate blood vessels (differentiation into vascular endothelial cells and vessel formation), the increase in the number of vascular endothelial precursor cells is preferably 1.2 times or more.

Here, peripheral blood refers to blood that flows through blood vessels located far from the heart. In addition, vascular endothelial precursor cells are stem cells that differentiate into vascular endothelial cells and are essential for vascularization.

Vascular endothelial precursor cells at an affected site differentiate from peripheral blood mononuclear cells (PBMnC), and have been confirmed to appear in one region of the map in flow cytometry analysis (Fluorescence Activated Cell Sorter; FACS), which uses the binding capacity between peripheral blood mononuclear cells and lectin and the uptake of dil-acetylated-low density lipoprotein (Dil-Ac-LDL) into mononuclear cells as indicators. Thus, increases in the number of vascular endothelial precursor cells can be confirmed by measuring the ratio of peripheral blood mononuclear cells present in that region.

The vascularization therapy of the present invention more preferably increases tissue blood flow volume by utilizing the vasodilator activity of carbon dioxide absorbed percutaneously, and/or inducing vascular growth factors by causing the pH of tissue fluid at the affected site to locally shift to the acidic side.

When an affected site is immersed in carbonated warm water containing a high concentration of carbon dioxide, the carbon dioxide is absorbed percutaneously, and the carbon dioxide concentration of the tissue increases. Thus, vasodilation occurs due to the body's regulatory mechanism, and tissue blood flow volume can be increased.

The absorbed carbon dioxide gradually reaches subcutaneous tissue, and then reaches residual vascular endothelial cells. Due to direct stimulation by this carbon dioxide , vascular growth factors are released from the endothelial cells. The absorbed carbon dioxide is ionized into carbonate ions within tissue fluid and as a result of causing the tissue to shift to the acidic side, the release of vascular growth factors is promoted.

In the method of the present invention, immersion of an affected site of a peripheral blood vessel in the aforementioned carbonated warm water is carried out continuously at a frequency of at least once every 24 hours, and the duration of a single immersion is preferably 10 minutes or more.

Since the vascularization therapy of the present invention involves stimulation of cells by carbon dioxide, it is preferably carried out intermittently over a long period of time.

The frequency of immersion is preferably at least once every 24 hours. Moreover, if the frequency of immersion is from 1 to 2 times every 24 hours, adequate effects are obtained and the load on the patient is not excessively large, thereby making this preferable.

In order to allow the action of the carbon dioxide to reach deeper, the immersion time of a single treatment is preferably at least 10 minutes. Moreover, if the duration of a single immersion is within the range of 10 to 30 minutes, adequate effects are obtained and the burden on the patient is not excessively large, thereby making this preferable.

In the case the aforementioned affected site of a peripheral blood vessel is accompanied by ulceration, it is preferable to dissolve antiseptic in the carbonated warm water during the process of immersing the affected site in the carbonated warm water.

Patients with peripheral vascular diseases frequently exhibit ulceration at the affected site. Thus, since there is concern over infection caused by bacteria in the water bath when using a carbonated warm water bath, it is preferable to immerse the affected site in carbonated warm water in which an antiseptic has been dissolved. The antiseptic should be that which does not cause the carbonated warm water to become alkaline while also having a low degree of irritation of the skin. Examples of such antiseptics include chlorhexidine glucuronate, povidone-iodine, dichlorocyanurates and hypochlorites.

In the case the aforementioned affected site of a peripheral blood vessel is accompanied by ulceration, the affected site is preferably immersed in carbonated warm water after applying a carbon dioxide-permeable film to the affected site.

As has been described above, in order to decrease the risk of bacterial infection, it is preferable to dissolve an antiseptic in the carbonated warm water. However, since some antiseptics have the action of inhibiting cellular growth, there are also cases in which therapeutic effects may be diminished.

In the present invention, since it is important to allow carbon dioxide to be absorbed through the skin from the carbonated warm water bath, preventing the passage of bacteria by applying a film permeable to carbon dioxide to the affected site can be carried out without reducing the effect of the carbon dioxide while preventing bacterial infection. Examples of such carbon dioxide permeable films include transparent dressings (such as Tegaderm (trade name) manufactured by 3M Health Care Ltd. or OpSite (trade name) manufactured by Smith & Nephew Co.) and silicon thin films having a high degree of gas permeability.

Although the dissolving of antiseptic in the carbonated warm water and the application of a carbon dioxide gas-permeable film to the affected site may be carried out independently, they may also be carried out by combining two types.

In the case the affected site of a peripheral blood vessel is located on a lower extremity and said disease is accompanied by pain of a lower extremity, it is preferable to immerse the site at least covering a range from below the articulatio genus to the aforementioned affected site in the aforementioned carbonated warm water in the aforementioned immersion step.

Although a carbonated warm water bath may be carried out only for an affected site of a lower extremity having ordinary subjective symptoms, since many of the arterial vessels to which the vascularization therapy of the present invention is preferably applied are located in muscle tissue, it is preferable to carry out the carbonated warm water bath so that the site of muscle located between the articulatio genus and the pedal is immersed in the carbonated warm water.

The vascularization therapy of the present invention is preferably carried out in combination with at least one type of vascularization therapy selected from direct administration of cell growth factor, introduction of a gene expressing cell growth factor and transplantation of bone marrow mononuclear cells (to be referred to as “other vascularization therapies” ).

Research is currently being conducted on treatment methods that promote vascular regeneration by injecting cell growth factors such as HGF (hepatocyte growth factor), VEGF (vascular endothelium growth factor) or FGF (fibroblast growth factor), or their expression genes, directly into an affected site as abovementioned. In addition, treatment methods have also been proposed in which vascular endothelial precursor cells and bone marrow mononuclear cells secreting the aforementioned growth factors are sampled from bone marrow followed by purification and transplantation to an affected area.

These vascularization therapies involving the direct administration of cell growth factors, introduction of genes expressing cell growth factors or transplantation of bone marrow mononuclear cells are preferably carried out intermittently in order to maintain the regenerated blood vessels. However, in addition to it being difficult to obtain the active substances that are administered, due to the considerable burden placed on the patient during administration, these therapies cannot be carried out repeatedly.

However, after carrying out these other vascularization therapies and the method according to the present invention simultaneously, or after carrying out these other vascularization therapies, by continuing to carry out the method according to the present invention, vascular function can be maintained over a long period of time without intermittently carrying out other vascularization therapies. In particular, since vascularization therapy by transplantation of bone marrow mononuclear cells temporarily demonstrates a high degree of efficacy, and does not require any special preparations, it is carried out preferably. However, since the obtaining of active substances from the bone marrow of the patient places a considerable burden on the patient, it is preferable to carry out the therapy of the present invention continuously following completion of transplantation of bone marrow mononuclear cells.

The vascularization action of the present invention is thought to be brought about due to a rise in carbon dioxide concentration in blood flowing through blood vessels. Normally, when the carbon dioxide concentration in the blood is attempted to be increased intentionally, it is accompanied by danger resulting from a simultaneous increase in carbon dioxide concentration in blood throughout the body. However, the carbonated warm water bath of the present invention only acts locally on an affected site, while also acting from outside vascular tissue. Thus, the desired action can be achieved while using a lower absolute amount of carbon dioxide . In addition, since excess carbon dioxide is rapidly eliminated by exhalation, the carbonated warm water bath of the present invention is extremely safe since there is no excessive increase in carbon dioxide concentration in blood throughout the body. Since carbon dioxide is inherently a substance that is widely dispersed throughout the body, there are considered to be no significant adverse side effects.

The vascularization therapy performed by the highly concentrated carbonated warm water bath of the present invention is associated with a high degree of safety with respect to the body, and can be performed easily without requiring any special complicated apparatuses or equipment.

Although the following provides an explanation of embodiments of the present invention, the scope of the present invention is not limited by these embodiments.

Preparation of Ischemia Model Mice and Immersion in Carbonated Warm Water

The left femoral artery and femoral vein were ligated in six-week-old male mice (C57BL/6J) to prepare a mouse model of lower extremity ischemia.

Animals of the carbonated warm water group consisted of ischemia model mice of which both legs were immersed in carbonated warm water (37° C.) having a carbon dioxide concentration of 1100 ppm for 10 minutes starting on day 4 after vascular ligation. Immersion was carried out once a day until the 28th day after model preparation. For the control group, with the exception of using distilled water (37° C.) not containing carbon dioxide, both legs of ischemia model mice were immersed in the same manner as animals of the carbonated warm water group.

The following effects were confirmed for animals of both the aforementioned carbonated warm water group and control group.

[1] Improvement of Blood Flow in Mice Ischemic Lower Extremities by Carbonated Warm Water Bath

In the present embodiment, the lower extremity blood flow of the ischemia model mice was measured. Lower extremity blood flow in the carbonated warm water group and control group was measured immediately after preparing the ischemia model and at 3, 7, 14, 21 and 28 days after model preparation using the Laser-Doppler Blood Perfusion Image Analyzer (Moor Instruments Inc.).

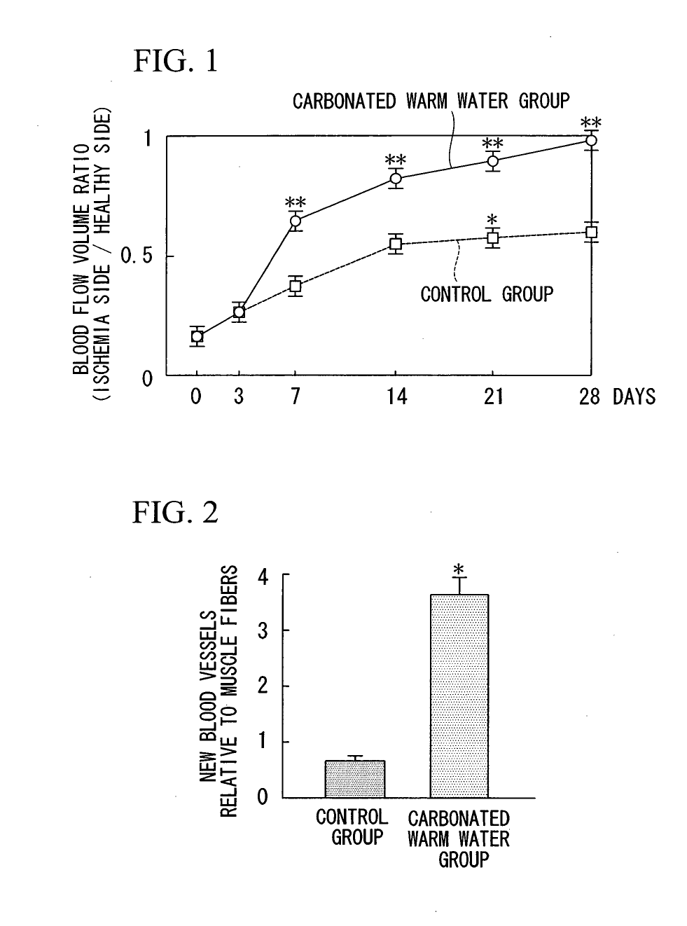

Those results are shown in FIG. 1. Quantification was carried out by measuring blood flow in the extremity on the healthy side not subjected to ischemia, and then indicating the blood flow volume ratio of the ischemic lower extremity relative to that of the healthy lower extremity.

As shown in FIG. 1, significant restoration of blood flow in the ischemic lower extremity was observed in the carbonated warm water group starting 7 days after model preparation. At 28 days after model preparation, a significant increase in blood flow volume of about 1.9 times was observed as compared with the control group.

[2] Increase in Vascularization in Mouse Ischemic Lower Extremity Skeletal Muscle by Carbonated Warm Water Bath

In the present embodiment, lower extremity angiography was performed by injecting contrast medium (Lipiodol, manufactured by Laboratoire Guerbet) into the mouse heart on day 28 after model preparation. A remarkable increase in collateral vessels was observed in the carbonated warm water group as compared with the control group by observing the resulting angiograms. In addition, the number of new blood vessels relative to muscle fibers was measured by staining mouse lower extremity skeletal muscle tissue with ALP. Those results are shown in FIG. 2.

The number of blood vessels increased by about 5.5 times in the carbonated warm water group as compared with the control group, thereby demonstrating a significant increase in the number of new blood vessels in the carbonated warm water group.

[3] Increase in Vascular Endothelial Cells in Mouse Ischemic Lower Extremity Skeletal Muscle by Carbonated Warm Water Bath

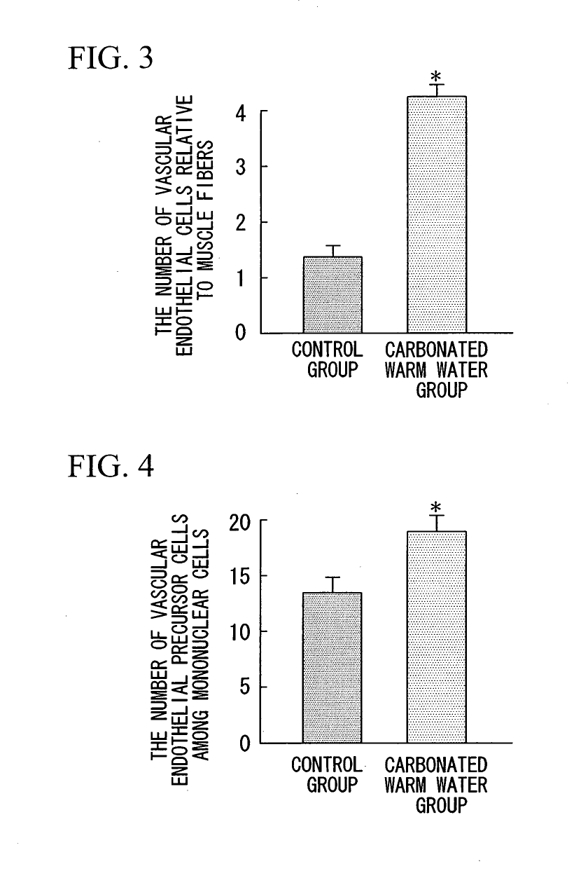

In the present embodiment, the number of vascular endothelial cells relative to muscle fibers was measured by immunostaining mouse lower extremity skeletal muscle using vWF factor antibody specific for vascular endothelium on day 28 after model preparation. Those results are shown in FIG. 3.

The number of vascular endothelial cells in the carbonated warm water group increased by about 3.1 times more than the control group, thereby demonstrating that vascularization was significantly promoted in the carbonated warm water group.

[4] Increase in Vascular Endothelial Precursor Cells in Mouse Peripheral Blood by Carbonated Warm Water Bath

In the present embodiment, the number of vascular endothelial precursor cells among peripheral blood mononuclear cells was measured by performing flow cytometry analysis (FACS) using as indicators the capacity of mouse peripheral blood mononuclear cells (PBMnC) to bind lectin and the uptake of dil-acetylated-low density lipoprotein (Dil-Ac-LDL) on day 28 after model preparation. Those results are shown in FIG. 4.

The number of vascular endothelial precursor cells increased by about 1.4 times in the carbonated warm water group relative to the control group, thereby demonstrating that vascularization was significantly promoted in the carbonated warm water group.

1. A vascularization therapy comprising the step of:
performing a vascularization treatment which increases the number of newly formed blood vessels of an affected site, the affected site of a peripheral blood vessel being immersed in carbonated warm water having a carbon dioxide concentration of at least 700 ppm and a temperature of 33 to 42° C.

2. A vascularization therapy comprising the step of:
performing a vascularization treatment which increases the number of vascular endothelial cells in tissue of an affected site by at least 1.5 times, the affected site of a peripheral blood vessel being immersed in carbonated warm water having a carbon dioxide concentration of at least 700 ppm and a temperature of 33 to 42° C.

3. A vascularization therapy that increases the number of endothelial precursor cells in the peripheral blood at an affected site by at least 1.1 times, comprising the step of performing a vascularization therapy in which the affected site of a peripheral blood vessel is immersed in carbonated warm water having a carbon dioxide concentration of at least 700 ppm and a temperature of 33 to 42° C.

4. A vascularization therapy according to any one of claims 1 through 3, wherein the number of new blood vessels at an affected site is increased by at least one of
increasing the tissue blood flow volume by the vasodilation action of carbon dioxide absorbed percutaneously,
or inducing vascular growth factors by causing the pH of tissue fluid at the affected site to become locally acidic.

5. A vascularization therapy according to any one of claims 1 through 3, wherein the therapy is performed intermittently at a frequency of once or more every 24 hours, and the duration of a single immersion is 10 minutes or more.

6. A vascularization therapy according to any one of claims 1 through 3, wherein an antiseptic is dissolved in the carbonated warm water in the step in which the affected site is immersed.

7. A vascularization therapy according to any one of claims 1 through 3, wherein the affected site of the peripheral vessel is accompanied by ulceration, and a carbon dioxide-permeable film is applied to the site of ulceration.

8. A vascularization therapy according to any one of claims 1 through 3, wherein the affected site of the peripheral vessel is located in lower extremities, the affected site is accompanied by lower extremity pain, and a site extending at least over the range from the articulatio genus to the affected site is immersed in the carbonated warm water in the immersion step.

9. A vascularization therapy according to any one of claims 1 through 3, wherein one or more steps are performed selected from
direct administration of cell growth factor,
introduction of gene that expresses cell growth factor, and
transplantation of bone marrow mononuclear cells.
